(19) United States
(12) Patent Application Publication
(10) Pub. No.: US 2002/0004214 A1
(43) Pub. Date: Jan. 10, 2002

(54) Method to detect modulators of histidine kinases

(76) Inventors: Raul Goldschmidt, Hartsdale, NY (US); Michael Loeloff, Summit, NJ (US)

Correspondence Address: Philip S. Johnson, Esq., Johnson & Johnson, One Johnson & Johnson Plaza, New Brunswick, NJ, 08933-7003, US

(21) Appl. No.: 09/733,731

(22) Filed: Dec. 8, 2000

Related U.S. Application Data
(60) Provisional application No. 60/172,924, filed on Dec. 21, 1999.

(51) Int. Cl. G01N 33/537; G01N 33/543; C07H 21/04; C12N 9/12

(52) U.S. Cl. 435/7.92; 435/194; 536/23.2

(57) ABSTRACT

The present invention provides methods to measure phosphorylation and transphosphorylation by histidine kinases proteins and assays to detect modulators of histidine kinase activity. The method of the invention is a robust, sensitive assay of simple design that is easily automated and an assay design that can be easily modified to allow different histidine kinase and response regulator targets to be tested without significant modification to the design. The present invention relates to scintillation proximity assays (SP) useful to screen compounds that modulate histidine kinase function.

### BACKGROUND OF THE INVENTION

1. Field of the Invention

The present invention relates to assays for the detection of compounds with pharmacological activity. More particularly, the invention relates to methods for the detection of modulators of histidine kinases, assays employing such methods and to compositions for use in such methods and assays.

2. Background

In order to survive bacteria need to adapt to their environments, a critical aspect of which is the ability to monitor their environmental changes in physical and chemical conditions. When they occur, bacteria generally react by shifting levels of expression of some of their genes. These changes result in the synthesis of proteins that allow bacteria to adapt to the new conditions. Adaptive responses in bacteria are often mediated by two-component signal-transducing systems (TCS). In their simplest form they consist of a sensory histidine protein kinase (HPK) and a response regulator (RR) effector (Parkinson and Kofoid, 1992). The HPK commonly span the bacterial cytoplasmic membrane. Upon sensing an environmental signal, the HPK autophosphorylates on a conserved histidine residue, and this phosphoryl group is transferred to a conserved aspartate on its cognate RR. The phosphorylated RR is then capable of regulating the transcription of one or more genes. Thus, a TCS acts by sensing and transducing an environmental signal to elicit a bacterial adaptive response.

TCS often control the expression of virulence traits among bacteria that cause infectious disease in humans. In addition, they are associated with the regulation of resistance mechanisms to β-lactams, polymyxin B, tetracycline, and vancomycin.

There are at least six features that make TCS attractive targets for the development of novel antimicrobial agents (Barrett et al., 1998): i) they are present in most bacterial species; ii) most bacteria contain multiple TCS, each generally controlling different functions; iii) in pathogenic bacteria TCS often regulate expression of virulence traits that are essential for their survival inside the host; iv) HPKs and also RRs display a high degree of homology around the active sites; v) they have not been found in either vertebrates or invertebrates (Alex and Simon, 1994); vi)X-ray crystallographic structures exist for a few HPKs and RRs. These properties suggest that it may be possible to find inhibitors of multiple TCS whose effect is to interfere with the adaptive responses of bacterial pathogens, and possibly inhibit their growth, without affecting the functions of their eukaryotic host.

A different facet of the potential of TCS as targets for the development for novel antimicrobials became apparent when it was discovered that some TCS are encoded by essential genes. This means that the TCS encoded by these genes are indispensable for cell survival, and their inactivation or the inhibition of their activity, cause bacterial death. Hence, TCS encoded by essential genes are particularly attractive as targets for antimicrobial targets.

The TCS genes espB and espA encoded for the HPK EspB and the RR EspA, respectively are described in PCT application WO9932657 entitled “Staphylococcus aureus Histidine Protein Kinase Essential Genes.” As described in W09932657, investigators at Microcide constructed a strain of S. aureus containing a conditional lethal mutation affecting the espA gene, and used this strain together with the isogenic wild type strain to establish a cell-based assay to screen for inhibitors of the EspB/EspA TCS.

Cell-based screens such as that described by Microcide fail to identify inhibitors of TCS unambiguously for at least two reasons. First, the cell permeability barrier may prevent access of some inhibitors to the cytoplasmic target. Second, because the TCS regulates other bacterial functions, inhibitors of the latter may be erroneously construed the activity of the TCS. Thus, a protein-based assay would not only allow a faster screen, but it would be desirable in order to ensure the sensitivity and specificity of the assay, for EspB and EspA. However no assay has yet been described.

Using scintillation proximity technology, homogeneous assays have been developed for a variety of molecular targets (Cook, N. D. (1996). Drug Discovery Today 1: 287-294; Picardo, M. and Hughes, K. T. (1997). In High Throughput Screening [Devlin, J. P. (Ed)], Dekker, New York, N.Y., pp. 307-316). Briefly, the target of interest is immobilized either by coating or incorporation on a solid support that contains a fluorescent material. A radioactive molecule, brought in close proximity to the solid phase by associating with the immobilized target, causes the fluorescent material to become excited and emit visible light. Emission of visible light forms the basis of detection of successful ligand/target interaction, and is measured by an appropriate monitoring device. An example of a scintillation proximity assay is disclosed in U.S. Pat. No. 4,568,649, issued Feb. 4, 1986. U.S. Pat. No. 5,770,176 describes assays for nuclear receptors wherein the functional receptor binds to immobilized nucleic acid. Materials for these types of assays are commercially available from DuPont NEN® (Boston, Mass.) under the trade name FlashPlate™. Development of scintillation proximity assays for the detection of kinase function has been described for serine/threonine kinases to peptide substrates (Naykayama, G.R. et al J. Biomolecular Screening (1998). 3: 43-48. McDonald, O. B. et al (1999). Anal Biochem 268: 318-329.) There are no reported scintillation proximity assays describing histidine kinase function or testing inhibitory compounds using similar techniques. Further, the assay techniques of serine threonine kinases are not easily adaptable to histidine kinases because, although both classes of enzymes are involved in the ultimate transfer of a phosphate to a receptive residue, because a) the mechanism of action of the different classes of enzymes is different; b) the phosphohistidinyl residue is unstable; and c) because of the latter, no antibodies specific to the phosphohistidinyl residue in histidine protein kinases has thus far been developed. Histidine kinases have a more complex mechanism requiring a phosphorelay system as previously described, while serine/threonine kinases act directly upon their substrate.

### SUMMARY OF THE INVENTION

The present invention provides methods to measure phosphorylation and transphosphorylation by histidine kinase proteins and assays to detect modulators of histidine kinase activity. The method of the invention is a robust, sensitive assay of simple design that is easily automated and an assay design that can be easily modified to allow different histidine kinase and response regulator targets to be tested without significant modification to the design. The present invention further relates to scintillation proximity assays (SP) useful to screen compounds that modulate histidine kinase function.

Thus the invention provides a method and assay for the detection of compounds that modulate histidine kinase enzymatic activity or modulate interaction of the kinase with its cognate response regulator protein comprising the steps of;

1) providing a compound, a histidine kinase, wherein the kinase has functional histidine kinase activity, and a histidine kinase substrate;

2) contacting the compound with the histidine kinase and the histidine kinase substrate;

3) isolating the histidine kinase substrate by affinity capture; and

4) detecting a change in kinase activity by monitoring the rate or absolute amount of phosphate transfer by the kinase to the substrate in the presence of the compound.

In a particular embodiment, there is described herein the design and use of a scintillation proximity-based assay for screening compounds that modulate the EspB kinase in two formats. One format is to measure autophosphorylation of EspB, and the second assay format measures transphosphorylation of its cognate response regulator protein, EspA. The methods described herein can easily be adapted for other histidine kinases and their cognate response regulator proteins. In a particular embodiment a solution containing radiolabeled ATP, a kinase protein and a target protein, are incubated for sufficient time to allow phosphorylation of the target protein. The target protein becomes immobilized via an affinity capture to the surface of the SP vehicle. Afterwards, the bound radiolabel is measured indirectly by monitoring the fluorescence of the immobilized support containing the immobilized target.

In one aspect the invention is further directed to a histidine kinase fusion protein for use in the assays of the present invention comprising a protein domain of a histidine kinase having functional catalytic activity fused or linked to a substance having affinity capture characteristics. The substance having affinity capture characterstics is one which is capable of being isolated or detected by binding to an affinity substrate. For example, the substance may be a protein or peptide having at least one affinity capture domain.

### BRIEF DESCRIPTION OF THE DRAWINGS

FIG. 1—Schematic of the Autophosphorylation Assay

FIG. 2—Schematic of the Cognate Response Regulator Assay

FIG. 3—Autophosphorylation in HTS format assay results

FIG. 4—Activity of MalE-EspB fusion protein in SDS-PAGE

FIG. 5—Transphosphorylation of EspA by EspB SDS-PAGE assay and effect of inhibitor

FIG. 6—Autophosphorylation of MalE-EspB protein fusion assay by SDS-PAGE.

### DETAILED DESCRIPTION

Definitions:

The term “protein domain” as used herein refers to a region of a protein that can fold into a stable three-dimensional structure, often independently of the rest of the protein, and which is endowed with a particular function. This structure may maintain a specific function associated with the domain's function within the original protein including enzymatic activity, creation of a recognition motif for another molecule, or provide necessary structural components for a protein to exist in a particular environment of proteins. Within a protein family and within related protein superfamilies protein domains can be evolutionarily conserved regions.

The term “protein superfamily” as used herein refers to sets of proteins whose evolutionary relationship may not be entirely established or may be distant by accepted phylogenetic standards, but show similar three dimensional structure or display a unique consensus of critical amino acids. The term “protein family” as used herein refers to proteins whose evolutionary relationship has been established by accepted phylogenic standards.

The term “fusion protein” as used herein refers to a novel chimeric protein construct that is the result of combining two or more domains or linker regions from different proteins for the purpose of combining in one single polypeptide chain functions and recognition properties normally associated with two or more distinct polypeptides. This is most often accomplished by the adjacent molecular cloning of the nucleotide sequences encoding for the desired protein domains to result in the creation of a new polynucleotide sequence that codes for the desired protein. Alternatively, creation of a fusion protein may be accomplished by chemically joining two proteins together.

The term “linker region” or “linker domain” or similar such descriptive terms as used herein refers to stretches of polynucleotide or polypeptide sequence that are used in the construction of a cloning vector or fusion protein. Functions of a linker region can include introduction of cloning sites into the nucleotide sequence, introduction of a flexible component or space-creating region between two protein domains, or creation of an affinity tag for specific molecule interaction. A linker region may be introduced into a fusion protein without a specific purpose, but as a compromise that results from choices made during cloning.

The term “cloning site” or “polycloning site” as used herein refers to a region of the nucleotide sequence contained within a cloning vector or engineered within a fusion protein that has one or more available restriction endonuclease recognition sequences. Adequate manipulation of restriction endonuclease sites allows the cloning in tandem of two or more nucleotide sequences so that the respective encoded protein domains are translated in frame relative to a particular start codon, thus yielding a desired protein product after transcription and translation. These nucleotide sequences can then be introduced into other cloning vectors, used to create novel fusion proteins, or used to introduce specific site-directed mutations. It is well known by those in the art that cloning sites can be engineered at a desired location by silent mutations, conserved mutation, or introduction of a linker region that contains desired restriction enzyme consensus sequences. It is also well known by those in the art that the precise location of a cloning site can be flexible so long as the desired function of the protein or fragment thereof being cloned is maintained.

As used herein, “expression vectors” are defined herein as nucleic acid sequences that are required for the transcription of cloned copies of genes and the translation of their mRNAs in an appropriate host. Such vectors can be used to express eukaryotic or prokaryotic genes in a variety of hosts including E. coli, blue-green algae, plant cells, insect cells, fungal cells including yeast cells, and animal cells.

As used herein, a “functional derivative” of the histidine kinase or its cognate response regulator protein is a construct that possesses a biological activity, either functional or structural, that is substantially similar to the properties described herein. The term “functional derivatives” is intended to include the “fragments,” “variants,” “degenerate variants,” “analogs” and “homologues” of the construct presented. The term “fragment” is meant to refer to any nucleic acid or polypeptide subset of the modules described herein. The term “variant” is meant to refer to a construct or coding sequence module substantially similar in structure and function to either the entire protein molecule or to a fragment thereof. A construct is “substantially similar” to the protein if both molecules expressed from them have similar structural characteristics or if both molecules possess similar biological properties. Therefore, if the two molecules possess substantially similar activity, they are considered to be variants even if the structure of one of the molecules is not found in the other or even if the two amino acid sequences are not identical. The term “analog” refers to a molecule substantially similar in function to either the entire protein molecule or to a fragment thereof.

The term “compound” as used herein in connection with a modulator of a histidine kinase protein refers to an organic or inorganic molecule that has the potential to disrupt the specific enzymatic activity of the kinase. For example, but not to limit the scope of the current invention, compounds may include small organic or inorganic molecules, synthetic or natural amino acid peptides, proteins, or synthetic or natural nucleic acid sequences, or any chemical derivatives of the aforementioned.

The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

The term “kinase” as used herein refers to members of the histidine kinase protein family or superfamily. Sources of kinase for use in the invention can be purified protein derived from natural or recombinant sources, with purified, recombinant protein being generally preferred. Histidine kinase substrates are either the histidine kinase itself, via an autophosphorylation mechanism, or a specific cognate response regulator protein, via a transphosphorylation mechanism.

The term “cognate response regulator” protein as used herein refers to non-kinase members of two-component systems. Cognate response regulator proteins suitable for use in the invention can include purified protein derived from natural or recombinant sources, with purified, recombinant protein being generally preferred. The cognate response regulator could also be a fusion protein or peptide that contains a recognition domain for the corresponding hisitidine kinase.

The term “high throughput” as used herein refers to an assay design that allows easy analysis of multiple samples simultaneously, capacity for robotic manipulation, and use of small sample volume. Examples of assay formats include 96-well or 384-well plates used for liquid handling experiments. It is well known by those in the art that as miniaturization of plastic molds and liquid handling devices are advanced, or as improved assay devices are designed, that greater numbers of samples may be performed using the design of the present invention. Use of 96-well plate assays in the examples is given for illustrative purposes only.

The term “target” as used herein refers to a protein that the histidine kinase phosphorylates and is captured by the scintillation proximity vehicle. The target protein may either be the kinase protein itself or its specific cognate response regulator.

The present invention provides assays to detect compounds that modulate histidine kinase enzymatic activity or modulate interaction of the kinase with its cognate response regulator protein. The method comprises the steps of;

1) providing a compound, a histidine kinase or functional derivative thereof, wherein the kinase has functional histidine kinase activity, and a histidine kinase substrate;

2) contacting the compound with the histidine kinase and the histidine kinase substrate;

3) isolating the histidine kinase substrate by affinity capture; and

4) detecting a change in kinase activity by monitoring the rate or absolute amount of phosphate transfer by the kinase to the substrate in the presence of the compound.

Two particular mechanisms to measure the amount of ATP consumed are either to monitor the exchange of a phosphate molecule from ATP to the histdine kinase (autophosphorylation) or from the histidine kinase to a substrate protein or peptide of the histidine kinase (transphosphorylation). Compounds that are agonists increase the rate or total amount of phosphate transferred to the histdine kinase substrate. Compounds that are antagonists decrease the rate or total amount of phosphate transferred to the histidine kinase substrate. The change in kinase activity is detected by isolation of the phosphate labeled protein or peptide by affinity capture of either the histidine kinase or the cognate response regulator protein and measuring the amount of radioactive phosphate directly or indirectly through a scintillation proximity vehicle.

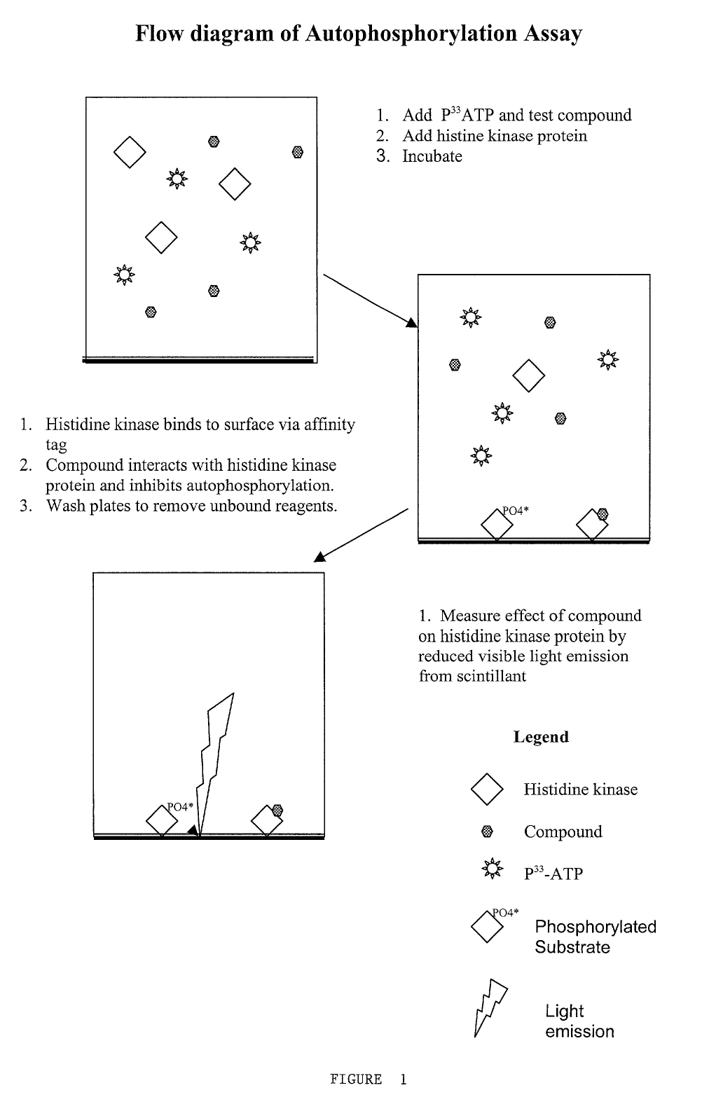

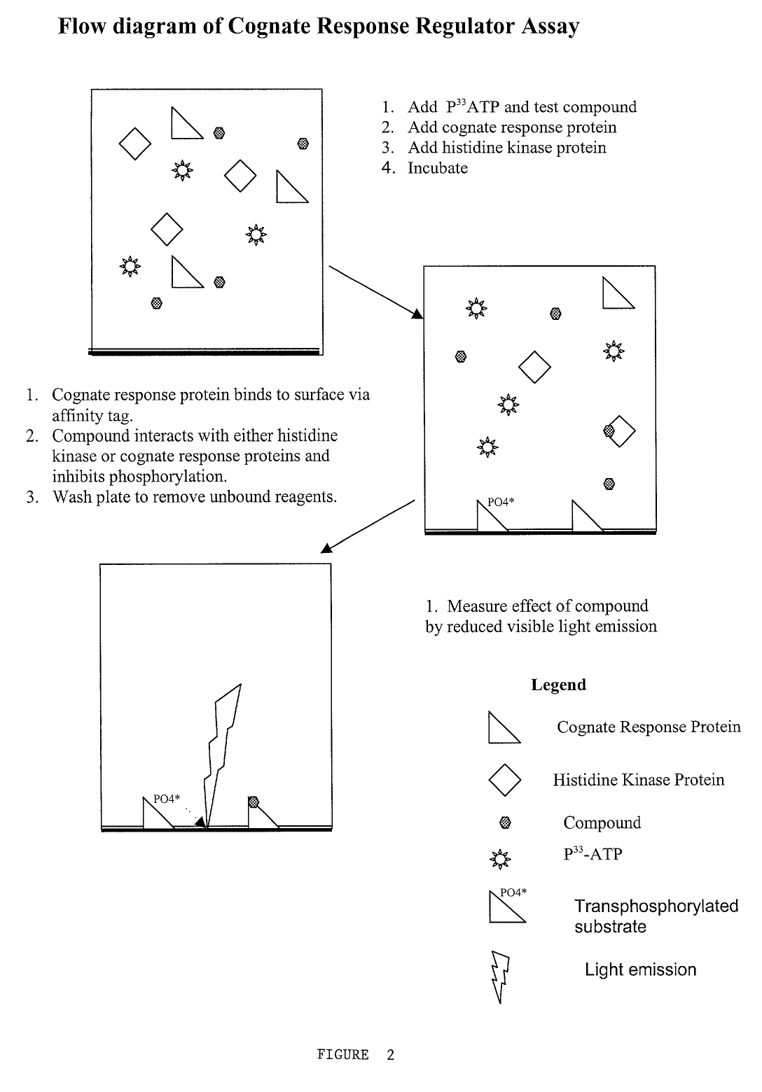

Two particular immunoaffinity capture assays of the invention are named Autophosphorylation Assay and Cognate Response Regulator Assay. In the Autophosphorylation Assay a protein, containing a histidine kinase catalytic domain and at least one affinity capture domain, is incubated in a buffer suitable for its enzymatic activity in the presence of radiolabeled ATP. The kinase protein autophosphorylates, resulting in the transfer of a radiolabeled phosphate group to a histidine amino acid residue, and simultaneously binds to the surface of the scintillation proximity vehicle through a matched affinity capture pair as illustrated in FIG. 1. The phosphorylation is measured by emission of visible light from the scintillation proximity vehicle. As illustrated in FIG. 2, in the Cognate Response Regulator Assay, a cognate response regulator protein containing a phosphorylation domain and at least one affinity capture domain is incubated in a buffer suitable for the matching kinase's enzymatic activity in the presence of radiolabeled ATP. The matched kinase protein is added to the mixture and phosphorylates the cognate response regulator protein, resulting in the transfer of a radiolabeled phosphate group to an aspartic acid residue to the cognate response regulator fusion protein. Simultaneously the cognate response regulator fusion protein binds to the surface of the scintillation proximity vehicle through a matched affinity capture pair. The phosphorylation is measured by emission of visible light from the scintillation proximity vehicle. In both of these cases, the affinity domain may be the existing protein domain or may be a distinct domain or small peptide introduced as part of a fusion protein.

In order to be sufficiently sensitive, a scintillation proximity assay requires efficient immobilization of the phosphorylated protein of interest to the scintillant-containing surface. Binding of target protein can be achieved by simple adsorption, but this does not usually yield a stably bound protein. While it is possible that adding greater amounts of target protein would increase the sensitivity of the reaction, this would create greater requirement for protein, and thus make the method cost prohibitive. To overcome this limitation, the target protein can be immobilized to the surface using an intermediate affinity capture motif. Methods of affinity capture include but not limited to, biotin/avidin technology, directed fusion protein interactions such as dextran/maltose binding domain or metal chelation/Histidine tag, and antibodies directed against the protein itself or against a fusion tag contained in a fusion protein.

An aspect of the present invention involves construction of histidine kinase fusion proteins that maintain catalytic activity, create at least one affinity capture domain, and eliminate a hydrophobic membrane-spanning domain contained within the native protein. The fusion protein produced by expression vectors in appropriate hosts is more easily purified, and maintains the catalytic region of the original protein against which modulating compounds may be directed. Another aspect of the invention involves construction of a cognate response regulator fusion protein that maintains its transphosphorylation domain and creates at least one affinity capture domain. Yet another aspect of the invention involves construction of a cognate response regulator fusion protein that maintains its transphosphorylation domain and maintains its DNA-binding domain, which could be used as an affinity-matched pair with an appropriate nucleic acid coated scintillation proximity vehicle.

Thus, one aspect of the invention is directed to a histidine kinase fusion protein comprising: a protein domain of a histidine kinase or functional derivative thereof having functional catalytic activity, fused to a protein or peptide molecule having at least one affinity capture domain. In a preferred embodiment, the histidine kinase is EspB and the protein domain consists of the polypeptide region of EspB comprising the 397 amino acid carboxy terminal. In another preferred embodiment, the histidine kinase is EspB and the protein domain consists of the polypeptide region comprising the 311 amino acid carboxy terminal. In a preferred embodiment, the affinity capture protein is selected from the malE gene of Escherichia coli, and the glutathione S-transferase encoding gene of Schistosoma japonicum. In this embodiment, the affinity capture protein is fused to the EspB protein domain at the N-terminus. In another embodiment, the fusion protein incorporates a histidine tail comprising six consecutive histidine residues to provide an alternative affinity capture mechanism. In yet another embodiment, the fusion protein incorporates a malE gene of Escherichia coli affinity capture domain at the N-terminus, the EspB kinase domain, and a hexahistidine tail at the C-terminus.

The term “EspB histidine kinase” as used herein refers to the espB gene of Staphylococcus aureus, orthologs of EspB, and paralogs of EspB. An EspB ortholog is a protein expressed by a gene isolated from other bacterial species that are homologous to the espB gene of Staphylococcus aureus. EspB paralogs are proteins expressed by Staphylococcus aureus or other bacteria that can transphosphorylate EspA.

Another aspect of the invention is directed to a cognate response regulator fusion proteins comprising a protein domain of a cognate response regulator that maintains functional transphosphorylation activity, fused to a protein or peptide molecule having at least one affinity capture domain. In a particular embodiment, the cognate response regulator is EspA and the affinity capture protein is malE.

The present invention provides for the use of any scintillant-impregnated or coated high throughput vessel, with multiple well plates (96 well, 384 well etc.) being generally preferred. Scintillant-containing plates can be purchased from Packard or NEN Life Sciences. Where tagged proteins are to be used, plates can be purchased pre-coated with streptavidin, or appropriate capture material (anti-Flag antibody, anti-glutathione S-transferase antibody, etc.) can be pre-coated to plates prior to incubation with the tagged protein. For purposes of Histidine tagged fusion proteins, one could use a scintillation proximity vehicle coated with a nickel surface. Pre-coating of antibodies can be performed by overnight incubation at 4° C. in appropriate buffers, using plates containing anti-species antibody or treated with, for example, poly-lysine.

The type of antibody used to establish an assay of this type may be either monoclonal (recognizing one epitope of its target protein) or polyclonal (recognizing multiple epitopes). The optimal antibody for a given antigen can be determined experimentally, because some antibodies may yield greater signal-to-noise ratios than others, depending on the epitope or epitopes of the antigen that each recognizes. Monoclonal and polyclonal antibodies are available from a number of commercial sources (Affinity BioReagents, Stressgen, Transduction Laboratories). Custom antibodies can be generated by the user according to standard procedures well known in the art (immunization of rabbits or mice with pure protein or peptide). The amount of antibody to be used per well will depend primarily on its avidity for the protein or protein fragment, and is well known by those in the art. Likewise, the positions of engineered affinity tags should be chosen so as not to disrupt kinase activity. However, for histidine kinases or their cognate response regulator proteins in general, use of an epitope outside the kinase or phosphorylation domains, or positioning of a tag amino-terminal or carboxy-terminal affinity tag, is unlikely to lead to disruption.

The present invention allows use of any buffer that maintains an appropriate pH and salt concentration to allow protein binding to the plate and for proper kinase enzymatic activity with it appropriate substrate. Inclusion of a divalent cation is required to maintain enzymatic activity, with manganese, magnesium, or calcium being generally preferred. Depending on the stability of any specific protein, the present invention allows buffer additives that enhance stability of the protein, and these conditions are well known by those in the art.

Kits comprising the described invention may be prepared for use in a variety of purposes including but not limited to forensic analyses, diagnostic applications, and epidemiological studies. Such a kit would comprise a compartmentalized carrier suitable to hold in close confinement at least one container. The carrier would further comprise reagents such as recombinant kinase protein and the cognate response regulator protein, as required, a scintillation proximity vehicle, suitable buffers or concentrates, and radiolabeled ATP.

Compounds identified according to the methods disclosed herein may be used alone at appropriate dosages defined by routine testing in order to obtain optimal inhibition of the histidine kinase activity to inhibit growth and kill bacteria that cause infectious disease, while minimizing any potential toxicity. In addition, co-administration or sequential administration of other agents may be desirable.

The following examples illustrate the present invention without, however, limiting the same thereto.

### EXAMPLE 1

Cloning the Segment of the espB Gene Encoding the Catalytic Region of the Polypeptide

EspB is an essential histidine kinase protein that comprises cytoplasmic, intramembrane, and extracellular regions. The cytoplasmic region of the EspB protein was determined by hydrophilicity and amphiphilicity analyses of the amino acid sequence of the translated open reading frame. Such analyses indicated that the polypeptide region comprehending the 397 carboxy-terminal amino acids would be suitable for retrieving the histidine kinase activity of the enzyme. Further, it was anticipated that the large intramembrane region could pose solubility problems for purification of the protein due to its high degree of hydrophobic character.

The cytoplasmic domain of EspB was expressed as a fusion protein with N-terminal and optionally with C-terminal extension domains for several reasons. These include to provide an affinity tag for the polypeptide, for ease of purification, to facilitate the design of a high throughput assay, and to increase the overall solubility of the protein. The C-terminal domain of the espB gene was fused to either the malE gene of Escherichia coli, or to the glutathione S-transferase encoding gene of Schistosoma japonicum, which were added to the N-terminus. To increase the likelihood of obtaining fusion proteins with histidine kinase activity, fusions of the respective proteins with two different size segments of espB were constructed. Optionally, the C-terminus of the fusion proteins were incorporated with a histidine tail consisting of six consecutive histidine residues in order to provide an alternative affinity purification or assay capture mechanism.

To construct MalE-EspB fusions we used the pMal™-c2 plasmid (New England BioLabs). Two amplicons of the espB gene were generated through PCR amplification of DNA from S. aureus strain COL using Pfu DNA polymerase (Stratagene) and pairs of primers described in Table 1 (below). The first amplicon, obtained using primers HKFM02 (SEQ.ID.NO.1) and HKBM02 (SEQ.ID.NO.6), encoded the 397 C-terminal amino acids of EspB, starting at Asp²¹². The two primers had BamH I and Hind III sites, respectively, to allow the cloning into the same sites of the vector maintaining the proper reading frame for the protein fusion. The second amplicon, obtained using primers HKFM03 (SEQ.ID.NO.2) and HKBM02 (SEQ.ID.NO.6), encoded the 311 C-terminal amino acids of EspB, starting at Met²⁹⁸. Similarly to the first case, the two primers had BamH I and Hind III sites, respectively, to allow the cloning into the same sites of the vector maintaining the proper reading frame for the protein fusion.

To construct GST-EspB fusions we used the pGEX-5X-1 plasmid (Pharmacia Biotech). Two amplicons of the espB gene were generated through PCR amplification of DNA from S. aureus strain COL using pairs of primers described in Table 1. The first amplicon, obtained using primers HKFG02 (SEQ.ID.NO.3) and HKBG02 (SEQ.ID.NO.7), encoded the 397 C-terminal amino acids of EspB, starting at Asp²¹². The two primers had BamHI and Xho I sites, respectively, to allow the cloning into the same sites of the vector maintaining the proper reading frame for the protein fusion. The second amplicon encoded the 310 C-terminal amino acids, starting at Ala²⁹⁹. Similarly to the first pair of primers, they had also BamH I and Xho I sites, respectively, to allow the cloning into the same sites of the vector maintaining the proper reading frame for the protein fusion.

In all cases, the blunt-ended amplicons were cloned first into plasmid pCR®-BLUNT (InVitrogen). From these constructs, the appropriate fragments were obtained by gel purification, after cutting adequately oriented fragments with either both BamH I and Hind III or with BamH I and Xho I restriction endonucleases. These fragments were then ligated into the appropriately cut vectors pMal™-c2 or pGEX-5X-1, and transformed into competent E. coli DH5α. cells according to the manufacture's instructions. The inserts were sequenced to ensure the correct reading frame and the preservation of the original sequence.

TABLE 1
PCR Primers for Cloning S. aureus Essential HPK Gene Components
EspB histidine kinase

| SEQ. ID. NO Forward: | |
|---|---|
| 1 HKFM03: | CCT GGA TCC GAT ATG CGT AAC CAG ACG GTC |
| 2 HKFM03: | CCT GGA TCC ATG GCG AAA GAA GAC ATC ATC |
| 3 HKFG02: | CCT GGA TCC CCG ATA TGC GTA ACC AGA CGG TC |
| 4 HKFG03: | CCT GGA TCC TGG CGA AAG AAG ACA TCA TC |
| 5 MEF21A02: | ACG GAT CCA TGA AAA CTG AAG AAG GTA AAC TGG T |
| SEQ. ID. No Backward: | |
| 6 HKBM02: | CTG AAG CTT ACA CTC ATC AAG ACG AGT AGT GC |
| 7 HKBG02: | CTG CTC GAG ACA CTC ATC AAG ACG AGT AGT GC |
| 8 HKB21A01: | AGC TCG AGT TCA TCC CAA TCA CCG TCT TC |

EspA cognate response regulator

| SEQ. ID. No Forward: | |
|---|---|
| 9 RRFN01: | CCT GGA TCC GAG GTT TAT GCA AAT GGC TAG |
| SEQ. ID. NO Backward: | |
| 10 RRBNS01: | CGA CTC GAG TAC ATT TCC AGT GTC TTG TGC |
| 11 RRBNL01: | CGA CTC GAG CTC ATG TTG TTG GAG GAA ATA TCC |

### EXAMPLE 2

Expression and Purification of malE-espB cDNA in E. coli Expression Vectors

Transformants were used to inoculate cultures for the production of MalE-EspB protein. Cultures may be grown in M9 or LB media, whose formulation is known to those skilled in the art.

The malE-espB fusion gene was transformed into DH5α cells for fusion protein expression. One inoculation loop with transformed cells was inoculated into 125 milliliters of LB medium in the presence of 100 ug/ml of ampicillin. The culture was incubated at 37° C. with shaking overnight. The next morning, 50 milliliters of this culture was added to 2 liters of LB medium containing 100 μg/ml ampicillin and cultured at 37° C. with shaking until the OD₆₀₀ reached between approximately 0.6. The cells were induced for MalE-EspB expression by adding IPTG (Isopropyl β-d-thiogalactopyranoside) to final concentration of 0.5 mM and incubated with shaking at 37° C. The cells were then harvested after 4 hours of culture. The media was centrifuged at 4000×g and then the pellet was isolated and stored at −80° C. for later purification.

### EXAMPLE 3

Purification of EspB Fusion Proteins Using MalE/Amylose Affinity Reagents

Buffer A is composed of 50 mM Tris-HCl pH 7.5, 200 mM NaCl, 1 mM DTT, 2 mM EDTA, 0.1 mM PMSF, 5% v/v Glycerol.

Buffer B is composed of 50 mM Tris-HCl pH 7.5, 200 mM NaCl, 1 mM DTT, 2 mM EDTA, 0.1 mM PMSF, 5% v/v Glycerol, 10 mM maltose.

Dialysis Buffer is composed of 25 mM Tris-HCl pH 7.5, 1 mM DTT, 5% v/v Glycerol

40 ml of Buffer A was used to resuspend a pellet of induced bacterial cells. Protease inhibitors (Boehringer Mannheim) were then added and the suspension was transferred to a pre-cooledfrench press, and the cells were disrupted by applying 25,000 psi of pressure The lysed cells were centrifuged at 4 C for 30 minutes at 50,000×g. The supernatant was applied to an affinity column (Pharmacia XK-16/20) packed with approximately 25 ml of amylose resin that had been equilibrated by running 5 volumes of buffer A at a rate of 1.5 ml/min. The column was washed with an additional 70 ml of buffer A until all unbound protein eluted. Then a gradient of 0-100% Buffer B was applied over a 60 minute period at a flow rate of 1.5 ml/min. Fractions (5ml) were collected during the course of the gradient, and the protein containing fractions were combined. An additional step of protein denaturation and refolding to improve autophosphorylation activity was added, as follows. Guanidine-HCl was added to the solution with gentle mixing to a final concentration of 0.5 to 2M, and the resulting solution was allowed to sit on ice for 5-10 minutes prior to beginning dialysis. The sample solution was dialyzed twice for 2 hours per change at 4° C. agains dialysis buffer. The final solution was divided into aliquots, frozen, and stored at −80° C. until further use.

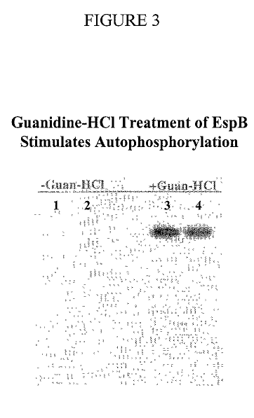

Enzyme autophosphorylation activity was assayed as described in Example 4 below. Samples containing equal amounts of purified MalE-EspB fusion that had either been treated or not with guanidine-HCl were incubated with [γ-³²P]ATP for 30 minutes, analyzed by polyacrylamide gel electrophoresis, and exposed to X-ray film. In FIG. 3 the bands show the autophosphorylated protein, and can be quantitated. Lanes 1 and 2 show the activity of purified protein samples that had not been pretreated with guanidine-HCl, whereas the protein samples inlanes 3 and 4 had been pretreated with 2 M and 0.5 M guanidine-HCl, respectively, and dialyzed afterwards against enzyme storage buffer. When the X-ray picture is greatly overexposed, the protein bands become also apparent on lanes 1 and 2. The experiment showes that pretreatment with guanidine-HCl activates MalE-EspB by approximately 40 to 100-fold, a likely consequence of denaturing and then refolding the purified MalE-EspB protein.

### EXAMPLE 4

EspB Autophosphorylation Gel Electrophoresis Assay

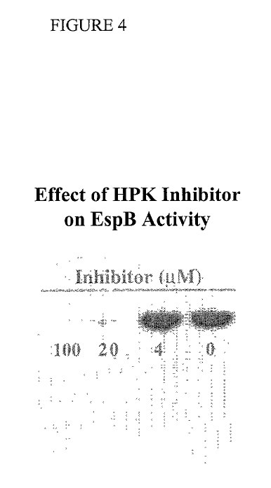

Volumes of 18.5 μl of compounds diluted in 6% DMSO or controls of 6% DMSO alone were mixed in a microcentrifuge tube with an equal volume of EspB reaction mixture (containing EspB at 0.58 mg/ml in a salt buffer of 328 mM Tris-HCl [pH 8.0], 225 mM KCl, 0.4 mM PMSF {phenylmethylsulfonyl fluoride}, 1.2 mM DTT, 0.2 mM EDTA, 9.3 mM MnCl₂, and 10% glycerol). Then 3 μl of ATP³² (containing a mixture of 667 mM of cold ATP and 66.7 mCi of [γ-³²P]ATP was added to each tube, resulting in a final concentration of 50 μM and an activity of 5 μCi ³²P. The complete reaction mixture was incubated for approximately minutes at ambient temperature, and was then stopped by adding 10 μl of SDS-PAGE loading buffer. The reaction was separated by SDS-PAGE electrophoresis (Hoefer, SE600 series electrophoresis unit) first, at 80 V through the stacker gel, followed by 250V through the separating gel. The dried gel was exposed to X-ray film. The bands show the MalE-EspB autophosphorylated protein and can be quantitated to determine the concentration of compound necessary to give 50% inhibition of the reaction (IC₅₀). In FIG. 4, the presence of an inhibitor completely eliminates any autophosphorylated espB band at 100 μM, substantially reduces the band at 20 μM and does not effect the band at 4 μM, compared to the 0 μM control band.

EspA Transphosphorylation by EspB Gel Electrophoresis Assay

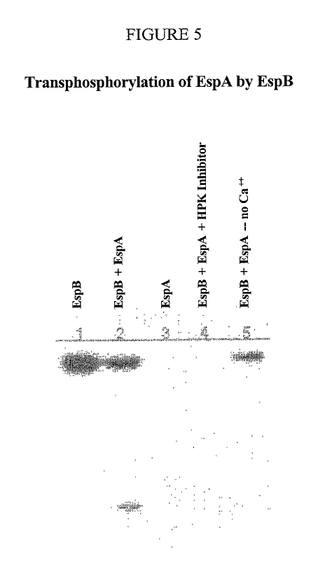

EspB with or without EspA, and EspA alone, were incubated with [γ-³²P]ATP for 30 minutes in a reaction with components in similar concentrations as described above with the following modifications. First, EspA was at a concentration of 0.58 mg/ml when present in the EspB reaction mixture. Second, Ca⁺⁺ (except as noted) was added as CaCl₂ at a concentration of 5 mM in the EspB reaction mixture. Experimental conditions and processing of the sqamples are similar to those described above. In FIG. 5, the autophosphorylated EspB fusion protein is seen as a band at the top of the gel (lanes 1, 2, and 5), whereas the transphosphorylated EspA protein is the band at the lower portion of the gel (see lane 2). The experiment in FIG. 5 shows that EspA is unable to autophosphorylate (lane 3), but that it can be transphosphorylated by its cognate HPK EspB (lane 2). FIG. 5 also shows that this transphosphorylation requires the presence of Ca⁺⁺ whereas autophosphorylation of EspB does not (absence of the EspA band in lane 5). Furthermore, FIG. 5 also shows that the reaction can be inhibited by an HPK inhibitor at 100 μM (an inhibitor of autophosphorylation), as indicated by the lack of any bands in lane 4. The protein bands can be quantitated to give the amount of autophosphorylation and transphosphorylation.

### EXAMPLE 5

Autophosphorylation Assay

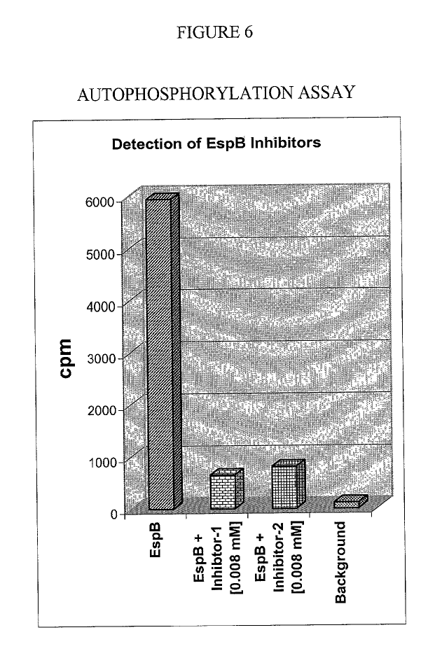

To the wells of an NEN Ni-coated FlashPlate™ were added sequentially (either manually or by robotics): 45 μl of Base reaction buffer (166.7 mM Tris-HCl [pH 8.0], 122.2 mM KCl, 0.111 mM DTT, 0.1 mM EDTA, and 4.4% glycerol); 2 μl of sample compounds prepared at a concentration of 0.4 mM in 30% DMSO and 50 mM HEPES [pH 7.4] (controls received the same amount of 30% DMSO and 50 mM HEPES [pH 7.4] with no compound); 2 μl of 125 mM MnCl₂ in 30% DMSO and 50 mM HEPES [pH 7.4]; 5 μl of EspB (MalE-EspB His-tagged) at 200 μg/ml in dialysis buffer (see example 3) (to all but background control wells); and 50 μl of 1 μCi/ml of [γ-³³P]ATP diluted in distilled deionized water (Milli-Q. The plates were incubated for a minimum of 45 minutes at room temperature. Thereafter all radioactivity unbound to the plates was removed with three sequential washes, each with 300 μl of 10 mM Tris-HCl. The fluorescence resulting from the ³³P decay electrons interacting with the interacting with the scintillant in the wells of the FlashPlate mwas measured using a TopCount scintillation counter. As shown in FIG. 6, two known inhibitors of HPK significantly reduced the EspB signal compared to the sample containing EspB alone. In this example, the His tag interaction with the NTA-Ni surface provided the affinity capture for the MalE-EspB-His-tagged fusion protien.

### EXAMPLE 6

Cognate Response Regulator Assay

To the wells of an NEN Ni-coated FlashPlate™ are added sequentially (either manually or by robotics): 45 μl of Base reaction buffer (166.7 mM Tris-HCl [pH 8.0], 122.2 mM KCl, 5.55 mM CaCl₂, 0.111 mM DTT, 0.1 mM EDTA, and 4.4% glycerol); 2 μl of sample compounds prepared at a concentration of 0.4 mM in 30% DMSO and 50 mM HEPES [pH 7.4] (controls receive the same amount of 30%DMSO and 50 mM HEPES [pH 7.4] with no compound); 2 μl of 125 mM MnCl₂ in 30%DMSO and 50 mM HEPES [pH 7.4]; 5 μl of EspB (MalE-EspB) at 200 μg/ml in dialysis buffer; 5 μl EspA (EspA-His tagged) at 200 μg/ml; and 50 μl of 1 μCi/ml of [γ-³³P]ATP diluted in distilled deionized water (Milli-Q. The plates are incubated for a minimum of 45 minutes at room temperature. Thereafter all radioactivity unbound to the plates is removed with three sequential washes, each with 300 μl of 10 mM Tris-HCl. The fluorescence resulting from the ³³P decay electrons interacting with the interacting with the scintillant in the wells of the FlashPlate™ is measured using a TopCount scintillation counter. Transphosphorylation of espA is measured by His capture of transphosphorylated espA. Compounds that inhibit either espB enzymatic activity, either autophosphorylation or transphosphorylation, will result in less transphosphorylated espA protein when compared to the control.

### REFERENCES

Alex, L. A., and Simon, M. I. (1994). Protein histidine kinases and signal transduction in prokaryotes and eukaryotes. Trends Genet. 10, 133-8.

Barrett, J. F., Goldschmidt, R. M., Lawrence, L. E., Foleno, B., Chen, R., Demers, J. P., Johnson, S., Kanojia, R., Fernandez, J., Bernstein, J., Licata, L., Donetz, A., Huang, S., Hlasta, D. J., Macielag, M. J., Ohemeng, K., Frechette, R., Frosco, M. B., Klaubert, D. H., Whiteley, J. M., Wang, L., and Hoch, J. A. (1998). Antibacterial agents that inhibit two-component signal transduction systems. Proc. Natl. Acad. Sci. U. S. A. 95, 5317-5322.

Parkinson, J. S., and Kofoid, E. C. (1992). Communication modules in bacterial signaling proteins. Annu. Rev. Genet. 26, 71-112.

What is claimed is:

1. A method of identifying compounds that modulate EspB histidine kinase enzymatic activity comprising the steps:
(a) admixing;
(i) a test compound,
(ii) an EspB histidine kinase fusion protein comprising an EspB histidine kinase catalytic domain and an affinity capture domain; and
(iii) a high energy phosphate source;
(b) allowing the compound, the histidine kinase fusion protein and the high energy phosphate source to incubate;
(c) isolating the EspB histidine kinase fusion protein by affinity isolation; and
(d) detecting a change in kinase activity by monitoring the rate or absolute amount of phosphate transfer to the EspB histidine kinase by autophosphorylation in the presence of the compound.

2. The method of claim 1 wherein the method is conducted in a single scintillant-impregnated or coated vessel and wherein the phosphorylated EspB histidine kinase is isolated by affinity capture onto the surface of the vessel.

3. The method of claim 1 wherein the affinity capture protein or peptide is selected from the group consisting of the malE gene of Escherichia coli, the glutathione S-transferase encoding gene of Schistosoma japonicum, and hexahistidine.

4. The method of claim 1 wherein the EspB histidine kinase catalytic domain comprises about the carboxy terminal 311 amino acids of the espB gene.

5. The method of claim 1 wherein the EspB histidine kinase catalytic domain comprises about the carboxy terminal 311 amino acids of the espB gene and the affinity capture domain is selected from the group consisting of malE gene of Escherichia coli, the glutathione S-transferase encoding gene of Schistosoma japonicum, and a hexahistidine sequence.

6. The method of claim 1 wherein the EspB histidine kinase catalytic domain comprises about the carboxy terminal 311 amino acids of the espB gene and the affinity capture domain is selected from the group consisting of malE gene of Escherichia coli, the glutathione S-transferase encoding gene of Schistosoma japonicum.

7. A method of identifying compounds that modulate histidine kinase enzymatic activity or modulate interaction of the kinase with its cognate response regulator protein comprising the steps:
(a) admixing;
(i) a test compound,
(ii) an EspA cognate histidine kinase or a functional derivative thereof, wherein the kinase has functional histidine kinase activity,
(iii) an EspA fusion protein comprising an EspA phosphorylation domain and an affinity capture domain; and
(iv) a high energy phosphate source;
(b) allowing the compound, the histidine kinase or derivative thereof, the EspA fusion protein and the high energy phosphate source to incubate;
(c) isolating the EspA fusion protein by affinity isolation; and
(d) detecting a change in kinase activity by monitoring the rate or absolute amount of phosphate transfer by the kinase to the EspA fusion protein in the presence of the compound.

8. The method of claim 7 wherein the method is conducted in a single scintillant-impregnated or coated vessel and wherein the EspA fusion protein is isolated by affinity capture onto the surface of the vessel.

9. The method of claim 7 wherein the affinity capture domain is selected from the malE gene of Escherichia coli, the glutathione S-transferase encoding gene of Schistosoma japonicum, and hexahistidine.

10. The method of claim 7 wherein the EspA cognate histidine kinase is EspB.

11. The method of claim 10 wherein the EspB histidine kinase is a fusion protein comprising about the carboxy terminal 311 amino acids of the espB gene.

12. The method of claim 7 wherein the EspA cognate histidine kinase is an ortholog of EspB.

13. The method of claim 7 wherein the EspA cognate histidine kinase is a paralog of EspB that can transphosphorylate EspA.

14. A histidine kinase fusion protein comprising a protein domain of the espB gene or functional derivative thereof having functional catalytic activity and a protein or peptide having at least one affinity capture domain.

15. The fusion protein of claim 14 wherein the protein domain comprises about the carboxy terminal 397 amino acids of the espB gene and an affinity capture domain selected from the group consisting of malE gene of Escherichia coli, the glutathione S-transferase encoding gene of Schistosoma japonicum, and a hexahistidine sequence.

16. The fusion protein of claim 14 wherein the protein domain comprises about the carboxy terminal 311 amino acids of the espB gene.

17. The fusion protein of claim 14 wherein the protein domain comprises about the carboxy terminal 311 amino acids of the espB gene and an affinity capture domain selected from the group consisting of malE gene of Escherichia coli, the glutathione S-transferase encoding gene of Schistosoma japonicum, and a hexahistidine sequence.

18. The fusion protein of claim 14 wherein the protein domain comprises about the carboxy terminal 311 amino acids of the espB gene and an affinity capture domain selected from the group consisting of malE gene of Escherichia coli and the glutathione S-transferase encoding gene of Schistosoma japonicum.


SEQUENCE LISTING (11 sequences)

SEQ ID NO: 1 - DNA, 30 bp, Artificial Sequence
  Description of Artificial Sequence Oligonucleotide
cctggatccgatatgcgtaaccagacggtc

SEQ ID NO: 2 - DNA, 30 bp, Artificial Sequence
  Description of Artificial Sequence Oligonucleotide
cctggatccatggcgaaagaagacatcatc

SEQ ID NO: 3 - DNA, 32 bp, Artificial Sequence
  Description of Artificial Sequence Oligonucleotide
cctggatccccgatatgcgtaaccagacggtc

SEQ ID NO: 4 - DNA, 29 bp, Artificial Sequence
  Description of Artificial Sequence Oligonucleotide
cctggatcctggcgaaagaagacatcatc

SEQ ID NO: 5 - DNA, 34 bp, Artificial Sequence
  Description of Artificial Sequence Oligonucleotide
acggatccatgaaaactgaagaaggtaaactggt

SEQ ID NO: 6 - DNA, 32 bp, Artificial Sequence
  Description of Artificial Sequence Oligonucleotide
ctgaagcttacactcatcaagacgagtagtgc

SEQ ID NO: 7 - DNA, 32 bp, Artificial Sequence
  Description of Artificial Sequence Oligonucleotide
ctgctcgagacactcatcaagacgagtagtgc

SEQ ID NO: 8 - DNA, 29 bp, Artificial Sequence
  Description of Artificial Sequence Oligonucleotide
agctcgagttcatcccaatcaccgtcttc

SEQ ID NO: 9 - DNA, 30 bp, Artificial Sequence
  Description of Artificial Sequence Oligonucleotide
cctggatccgaggtttatgcaaatggctag

SEQ ID NO: 10 - DNA, 30 bp, Artificial Sequence
  Description of Artificial Sequence Oligonucleotide
cgactcgagtacatttccagtgtcttgtgc

SEQ ID NO: 11 - DNA, 33 bp, Artificial Sequence
  Description of Artificial Sequence Oligonucleotide
cgactcgagctcatgttgttggaggaaatatcc